(19) United States
(12) Patent Application Publication
(10) Pub. No.: US 2026/0306575 A1
(43) Pub. Date: Oct. 8, 2026

(54) COMPOSITION FOR PREVENTING OR TREATING CANCER COMPRISING FERMENTED EXTRACT OF STEWARTIA PSEUDOCAMELLIA AS ACTIVE INGREDIENT

(71) Applicant: PUREBIO CO., LTD., Gyeongsangnam-do (KR)

(72) Inventor: Woo Hyun NAM, Seoul (KR)

(21) Appl. No.: 19/485,100

(22) PCT Filed: May 13, 2024
(86) PCT No.: PCT/KR2024/006465

§ 371 Date: Nov. 17, 2025

(30) Foreign Application Priority Data
May 19, 2023 (KR) 10-2023-0065282

(51) Int. Cl. A61K 36/82; A61P 35/00

(52) U.S. Cl. CPC A61K 36/82; A61P 35/00; A61K 2236/19; A61K 2236/331; A61K 2236/37

(57) ABSTRACT

The present application provides a composition for preventing and treating cancer, comprising a Stewartia pseudocamellia extract as an active ingredient.

### TECHNICAL FIELD

The present application claims priority and the benefit of Korean Patent Application No. 10-2023-0065282 filed on May 19, 2023 in the Korean Intellectual Property Office, the entire disclosure of which is incorporated herein by reference.

The present application relates to a method for preparing a composition for preventing or treating cancer, including a Stewartia pseudocamellia extract as an active ingredient.

### BACKGROUND ART

Cells undergo the processes of growth, differentiation, programmed death (apoptosis), or remain in a growth-arrested state, and these processes are strictly regulated. However, in the case of cancer cells, abnormalities occur in some of the genes of the cells, so that the properties of the proteins that are the products of these genes are changed, resulting in abnormalities in the regulation of cell growth. Since such abnormalities in the regulation of cell growth are accompanied by gene mutations, cancer is a genetic disease caused by abnormalities in genes.

When normal cells are exposed to risk factors that cause genetic mutations, they transform into cancer cells, and thus cancer develops. When normal cells are exposed to smoking, carcinogenic foods and chemicals, carcinogenic pathogens, and the like, which are known to be common risk factors for the development of cancer, genetic mutations occur. In addition, approximately 10 to 20% of cancer cases are genetically affected by abnormalities in genes inherited from parents. One of the components of the cell nucleus is a substance called DNA, and cancer cells are produced when the structure of this DNA changes. These altered cells continue to have altered DNA even as they divide, and eventually these cells continue to divide and multiply, leading to the development of cancer. These changes are not temporary, but rather, various types of genetic mutations accumulate over a period of 20 to 30 years, resulting in the development of cancer.

The normal human immune system has the ability to destroy up to 10 million tumor cells produced in the body. However, when cancer cells divide and proliferate to the extent that cancer is clinically detected, at least one billion tumor cells are usually included, significantly exceeding the level at which cancer cells can be destroyed by the immune system. Therefore, cancer cells are not eliminated and cancer develops.

Cancer is one of the incurable diseases that humanity must address, and even though huge capital is being invested in the development for curing cancer worldwide and medical technology is also being innovatively developed, the incidence of cancer and deaths from cancer continue to increase. According to recent statistics from the World Health Organization, it is known that about 10 million or more new cancer patients are diagnosed each year worldwide, and in Korea, cancer is also the leading cause of death, and each year, about 100,000 or more people are diagnosed with cancer and about 60,000 or more die from cancer. In particular, colorectal cancer is one of the most common malignant tumors in Korea, ranking second in incidence among carcinogenic diseases, and is showing a continuous upward trend.

There are various factors that cause cancer, and these factors can be divided into internal factors such as genetic factors or immunological factors, and external factors such as chemicals, radiation, or viruses. In particular, in relation to the recent increase in cancer patients, it is known that environmental factors account for a greater proportion of cancer incidence in modern society than genetic factors, and in particular, the cancer rate caused by diet is known to be extremely high, accounting for 36% of the cancer incidence due to environmental factors. Specifically, in the case of colorectal cancer, it is known that the main causes are increased intake of animal fats, sugar, and alcohol, and decreased intake of fiber, vegetables, fruits, and antioxidant vitamins.

Therefore, as long as the living environment in modern society remains fundamentally unchanged, the increasing trend in the incidence of cancer and the number of deaths from cancer is likely to continue. In particular, the importance of cancer prevention and early treatment is being emphasized even more in light of the fact that it is almost impossible to cure cancer when is not detected early, the more the disease progresses, the greater the suffering and economic losses for patients and their families become, and the greater the social costs, such as the depletion of medical insurance funds.

### DISCLOSURE

### Technical Problem

A problem to be solved by the present application is to provide a method for preparing a pharmaceutical composition for preventing or treating cancer, including a Stewartia pseudocamellia fermented product as an active ingredient.

A problem to be solved by the present application is to provide a pharmaceutical composition for preventing or treating cancer, including a lactic acid bacteria fermented extract of Stewartia pseudocamellia as an active ingredient.

A problem to be solved by the present application is to provide a food composition for preventing or ameliorating cancer, including a lactic acid bacteria fermented extract of Stewartia pseudocamellia as an active ingredient.

A problem to be solved by the present application is to provide a feed composition for preventing or ameliorating cancer, including a lactic acid bacteria fermented extract of Stewartia pseudocamellia as an active ingredient.

A problem to be solved by the present application is to provide a quasi-drug composition for preventing or ameliorating cancer, including a lactic acid bacteria fermented extract of Stewartia pseudocamellia as an active ingredient.

A problem to be solved by the present application is to provide a method for preventing or treating cancer, the method including administering a pharmaceutical composition including a Stewartia pseudocamellia fermented product as an active ingredient to an individual other than an individual.

A problem to be solved by the present application is to provide a use of a composition including a Stewartia pseudocamellia fermented product as an active ingredient for preventing or treating cancer.

The problems to be solved by the present application are not limited to those mentioned above, and other problems that are not mentioned may be clearly understood by those skilled in the art from the following description.

### Technical Solution

To solve the above-described problems, one embodiment of the present application provides a method for preparing a pharmaceutical composition for preventing or treating cancer, the method including preparing a Stewartia pseudocamellia extract; and inoculating the extract with a lactic acid bacteria culture solution and fermenting the extract to obtain a fermented product, wherein the pharmaceutical composition includes the fermented product as an active ingredient.

In one embodiment of the present application, the fermented product is obtained by fermenting a hydrothermal extract with lactic acid bacteria, and the lactic acid bacteria may be at least one selected from the group consisting of Lactobacillus plantarum, Lacticaseibacillus rhamnosus, and Lactiplantibacillus pentosus.

In one embodiment of the present application, in the obtaining of the fermented product, the fermentation may be performed at 30° C. to 37° C. for 1 day to 6 days.

In one embodiment of the present application, the preparing of the Stewartia pseudocamellia extract may extract Stewartia pseudocamellia by mixing a Stewartia pseudocamellia powder and a solvent in a ratio of 1:5 to 1:30.

In one embodiment of the present application, the Stewartia pseudocamellia may be one or more of leaves and stems, and the extract may be an extract extracted with hot water at 80° C. to 110° C. for 2 to 12 hours.

In one embodiment of the present application, the cancer may be at least one selected from the group consisting of breast cancer, pancreatic cancer, colorectal cancer, ovarian cancer, gastric cancer, cervical cancer, thyroid cancer, parathyroid cancer, lung cancer, non-small cell lung cancer, prostate cancer, gallbladder cancer, biliary tract cancer, bladder cancer, kidney cancer, melanoma, colon cancer, bone cancer, skin cancer, head and neck cancer, uterine cancer, rectal cancer, brain cancer, perianal cancer, fallopian tube carcinoma, endometrial carcinoma, vaginal cancer, vulvar carcinoma, esophageal cancer, small intestine cancer, endocrine gland cancer, adrenal cancer, soft tissue carcinoma, urethral cancer, penile cancer, ureteral cancer, renal cell carcinoma, renal pelvis carcinoma, blood cancer, acute myelogenous leukemia, myelodysplastic syndrome, chronic myelogenous leukemia, polycythemia vera, essential thrombocytosis, myelofibrosis, acute lymphoblastic leukemia, Hodgkin lymphoma, non-Hodgkin lymphoma, multiple myeloma, chronic lymphoblastic leukemia, central nervous system (CNS) tumors, spinal cord tumors, brainstem glioma, and pituitary adenoma.

One embodiment of the present application provides a pharmaceutical composition for preventing or treating cancer, which is prepared by the above preparation method and includes a lactic acid bacteria fermented extract of Stewartia pseudocamellia as an active ingredient.

One embodiment of the present application provides a food composition for preventing or ameliorating cancer, including a lactic acid bacteria fermented extract of Stewartia pseudocamellia as an active ingredient.

One embodiment of the present application provides a feed composition for preventing or ameliorating cancer, including a lactic acid bacteria fermented extract of Stewartia pseudocamellia as an active ingredient.

One embodiment of the present application provides a quasi-drug composition for preventing or ameliorating cancer, including a lactic acid bacteria fermented extract of Stewartia pseudocamellia as an active ingredient.

One embodiment of the present application provides a method for preventing or treating cancer, the method including administering a pharmaceutical composition including a Stewartia pseudocamellia fermented product as an active ingredient to a human.

One embodiment of the present application provides a method for preventing or treating cancer, the method including administering a pharmaceutical composition including a Stewartia pseudocamellia fermented product as an active ingredient to an individual other than a human.

One embodiment of the present application provides a use of a composition including a lactic acid bacteria fermented extract of Stewartia pseudocamellia as an active ingredient for preparing a drug for preventing or treating cancer.

One embodiment of the present application provides a use of a composition including a lactic acid bacteria fermented extract of Stewartia pseudocamellia as an active ingredient for preparing a food for preventing or treating cancer.

One embodiment of the present application provides a use of a composition including a lactic acid bacteria fermented extract of Stewartia pseudocamellia as an active ingredient for preparing a feed for preventing or treating cancer.

One embodiment of the present application provides a use of a composition including a lactic acid bacteria fermented extract of Stewartia pseudocamellia as an active ingredient for preparing a quasi-drug for preventing or treating cancer.

### Advantageous Effects

The composition according to one embodiment of the present application has an effect of killing cancer cells.

### DESCRIPTION OF DRAWINGS

FIG. 1 illustrates a cancer cell killing effect on blood cancer cells (Molt-4).

FIG. 2 illustrates a cancer cell killing effect on colorectal cancer cells (HT-29).

FIG. 3 illustrates a cancer cell killing effect on breast cancer cells (MCF-7).

FIG. 4 illustrates a cancer cell killing effect on pancreatic cancer cells (MIA PaCa-2).

### BEST MODE

Hereinafter, the present application will be described in more detail.

The following specific structural or functional descriptions are merely illustrative examples for describing embodiments according to the concept of the present application, and the embodiments according to the concept of the present application may be embodied in various forms and should not be construed as being limited to the embodiments described in the present specification.

Since the embodiments according to the concept of the present application may be variously modified and take various forms, specific embodiments will be described in detail in the present specification. However, this is not intended to limit the embodiments according to the concept of the present application to specific disclosed forms, and it is to be understood that all the changes, equivalents and substitutions included in the spirit and scope of the present application are included in the present invention.

The terms used in the present specification are used only to describe specific embodiments, and are not intended to limit the present application. Singular expressions include plural expressions unless the context clearly indicates otherwise.

Unless defined otherwise, all terms used herein, including technical or scientific terms, have the same meaning as commonly understood by a person with ordinary skill in the art to which the present application pertains. Terms such as those defined in commonly used dictionaries should be interpreted as having a meaning consistent with the meaning in the context of the relevant art and should not be interpreted in an ideal or overly formal sense unless explicitly defined in the present specification.

As used herein, the term “including as an active ingredient” refers to including an amount sufficient to affect a desired biological effect, such as a beneficial outcome including, but not limited to, prevention, reduction, relief, or elimination of signs or symptoms of a disease or disorder, and this amount is referred to as an “effective amount.” Therefore, the total amount of each active ingredient of the composition or additive is sufficient to exhibit a significant individual benefit. Accordingly, the effective amount will vary depending on the circumstances in which it is administered.

As used herein, the term “amelioration” refers to all actions that reduces a parameter associated with a condition to be treated by ingestion of the composition, for example, the severity of symptoms.

As used herein, the term “prevention” refers to blocking the appearance of a disease or symptoms associated with a disease in an asymptomatic subject who is at risk of developing the disease due to exposure to a disease cause. In addition, the term refers to all actions that suppress a disease or delay the onset of the disease by administering the composition of the present application.

As used herein, the term “treatment” refers to an approach for obtaining beneficial or desired clinical outcomes, and for the purposes of the present invention, includes, but is not limited to, alleviation of symptoms, reduction in the extent of disease, stabilized (that is, not worsening) state of disease, delaying or slowing the rate of disease progression, amelioration, palliation or relief of disease state, whether the beneficial or desired clinical outcome is perceptible or not, and whether partially or totally.

Therefore, the treatment refers to both therapeutic treatment and prophylactic measures, and what is to be treated includes not only conditions in which the disease is already present, but also conditions in which disease is to be prevented. Furthermore, the treatment may refer to all actions that improve or benefit the symptoms of cancer by administering the pharmaceutical composition of the present invention to an individual.

Stewartia pseudocamellia Maxim. is a deciduous tree of the Theaceae family. It is distributed in Jeollanam-do, Jeollabuk-do, Gyeongsangnam-do and Gyeongsangbuk-do in South Korea and mainly grows in the Jirisan area, where it is relatively abundant. This plant grows to a height of 7 to 15 m, its bark is reddish-brown with a hint of black, but parts of the bark peel off in round or long sections, revealing reddish-yellow patterns. Alternately arranged leaves are oval-shaped, and leaf margins have small serrations. Its flowers are white and bloom at the base of the leaves in June or July. The flowers are polypetalous flowers with five petals and many stamens. Its hard fruit ripening in October is covered with soft hairs. Since the bark has a mottled pattern, the plant is also called Korean mountain camellia or Stewartia koreana Nakai. Since the leaves turn yellow in autumn and the flowers are shaped like camellia flowers, the plant is often planted as an ornamental tree in gardens or parks. The wood is hard and often used for furniture, and currently, the plant is sought after by many people for its valuable medicinal properties and drinkable tea. The bark falls off in flakes, and has reddish-brown, grayish-white, and grayish-brown patterns, a liquid grows from the base of new branches, and the fruit is a pentagonal, ovate capsule. This plant is known by the synonym Stewartia koreana Nakai or Stewartia pseudocamellia Maxim. var. koreana (Nakai) Kim, and some consider the plant to be a unique species due to its cup-shaped flowers and straight and rounded branches, but it is mainly recorded as Stewartia pseudocamellia Maxim., which is currently distributed in Japan and other countries, and another synonym recorded is Stewartia ptero-petiolata Cheng var. koreana (Rehd.) Sealy. Stewartia pseudocamellia is used for ornamental purposes due to its large and showy flowers, and is used as furniture, a decorative material, and the like due to its hard and beautiful patterns, and the bark and root bark are called peony (cap orchid) and are effective in relaxing muscles and promoting blood circulation, and have been used to relieve blood stasis caused by bruises, and related activities associated with rheumatism have also been studied.

In the present application, a Stewartia pseudocamellia extract may be extracted from various organs of the natural, hybrid or variant plants of Stewartia pseudocamellia, and can be extracted from seeds, roots, aerial parts, stems, leaves, flowers, fruit bodies, and fruit peels of Stewartia pseudocamellia, as well as plant tissue cultures. Without being limited thereto, the Stewartia pseudocamellia extract may be one or more of a Stewartia pseudocamellia stem extract and a Stewartia pseudocamellia leaf extract.

As used herein, the term “extract” is commonly used in the art to mean a crude extract, but also includes a fraction obtained by additionally fractionating an extract in a broad sense. That is, the extract refers to a resulting product such as a liquid component obtained by immersing a desired substance in various solvents and then extracting the desired substance in a room temperature, low temperature, or high temperature state for a predetermined time, or a solid component obtained by removing the solvent from the liquid component. Furthermore, in addition to the resulting product, the term can be comprehensively interpreted to include all of the dilutions of the resulting product, concentrates thereof, crude purifications thereof, purified products thereof, and the like.

For example, a fraction obtained by allowing the extract to pass through an ultrafiltration membrane with a certain molecular weight cutoff value, and a fraction obtained through various purification methods additionally performed, such as separation by chromatography are also included in the extract of the present application.

In the present application, the method for extracting a Stewartia pseudocamellia extract is not particularly limited, and the Stewartia pseudocamellia extract may be extracted by a method typically used in the art. The extraction method includes hydrothermal extraction, ultrasonic extraction, filtration, reflux extraction, and the like, and these may be carried out alone or in combination of two or more thereof, but the extraction method is not limited thereto.

The type of extraction solvent used for extraction in the present application is not particularly limited, and any solvent known in the art may be used. As the extraction solvent, it is possible to use water or distilled water; an alcohol having 1 to 4 carbon atoms, such as methanol, ethanol, propyl alcohol, and butyl alcohol; a polyhydric alcohol such as glycerin, butylene glycol, and propylene glycol; and a hydrocarbon-based solvent such as methyl acetate, ethyl acetate, acetone, benzene, hexane, diethyl ether, and dichloromethane; or a mixture thereof, but the extraction solvent is not limited thereto. In addition, a solvent extract may be prepared by extracting the extract using the solvent one or more times, and a dried extract obtained by distilling the solvent extract under reduced pressure and then freeze-drying or spray-drying the resulting product may be prepared.

Specifically, water may be used as a solvent for the preparation of the extract in the present application.

In one embodiment of the present application, the preparing of the Stewartia pseudocamellia extract may extract the extract by mixing a Stewartia pseudocamellia powder and a solvent in a ratio of 1:5 to 1:30, and specifically, may extract the extract by mixing a Stewartia pseudocamellia powder and a solvent in a ratio of 1:5 or more, 1:7 or more, 1:10 or more, 1:12 or more, and 1:15 or more, and 1:30 or less, 1:29 or less, 1:28 or less, 1:27 or less, 1:26 or less, and 1:25 or less. More specifically, the preparing of the Stewartia pseudocamellia extract may extract the extract by mixing a Stewartia pseudocamellia powder and a solvent in a ratio of 1:13, 1:14, 1:15:1:16, 1:18, 1:20, 1:22, and 1:24.

The extraction temperatures may be 80° C. or higher, 82° C. or higher, 83° C. or higher, 84° C. or higher, 85° C. or higher, 86° C. or higher, 87° C. or higher, 88° C. or higher, 89° C. or higher, 90° C. or higher, 91° C. or higher, 92° C. or higher, 93° C. or higher, 94° C. or higher, 95° C. or higher, 120° C. less, 119° C. or less, 118° C. or less, 117° C. or less, 116° C. or less, 115° C. or less, 114° C. or less, 113° C. or less, 112° C. or less, 111° C. or less, 110° C. or less, 109° C. or less, 108° C. or less, 107° C. or less, 106° C. or less, 105° C. or less, and 96° C., 97° C., 98° C., 99° C., 100° C., 101° C., 102° C., 103° C., and 104° C.

The extraction time may be 2 hours or more, 3 hours or more, 4 hours or more, 5 hours or more, 12 hours or less, 10 hours or less, 8 hours or less, or 7 hours or less.

According to such extraction conditions, the component exhibiting the effect desired in the present invention may be efficiently extracted from the raw materials.

The prepared extract can be filtered or the solvent can be removed by performing a concentration or drying process, or all of the filtration, concentration and drying can be performed. For example, a filter paper may be used or a vacuum filter may be used for filtration, a vacuum concentrator may be used for concentration, and a freeze-drying method, and the like may be used for drying, but are not limited thereto.

As used herein, the term “fraction” refers to a product obtained by a fractionation method of separating a specific component or specific group from a mixture including various components.

In the present application, the fraction may be interpreted as a fraction obtained by subjecting the Stewartia pseudocamellia extract to various fractionation methods. The fraction of the extract may be obtained by subjecting the extract to various fractionation methods, and the fractionation method may be a solvent fractionation method performed by treatment with various solvents, an ultrafiltration fractionation method performed by allowing the extract to pass through an ultrafiltration membrane with a certain molecular weight cutoff value, a chromatographic fractionation method performed using various chromatographic techniques (designed for separation according to size, charge, hydrophobicity, or affinity), and the like.

In particular, the solvent used in the solvent fractionation is not particularly limited, but a polar solvent or a non-polar solvent may be used, and specifically, a non-polar solvent may be used. The solvent fractionation method may be performed by sequentially fractionating the extract using solvents with a high level of non-polarity to a low level of non-polarity, and for example, a method of sequentially fractionating the extract using acetic acid or ethyl acetate may be used. As the fractionation solvent, it is possible to use a polar solvent such as water, distilled water, or alcohol; or a non-polar solvent such as hexane, ethyl acetate, chloroform, or dichloromethane, and these solvents may be used alone or in combination of two or more, but are not limited thereto.

The present application provides a method for preparing a pharmaceutical composition for preventing or treating cancer, including a Stewartia pseudocamellia fermented product as an active ingredient.

The present application provides a pharmaceutical composition for preventing or treating cancer, including a lactic acid bacteria fermented extract of Stewartia pseudocamellia as an active ingredient.

The pharmaceutical composition may include a Stewartia pseudocamellia fermented product or a fraction thereof.

As used herein, the term “fermentation” refers to the decomposition of organic matter using enzymes possessed by microorganisms. In the present application, the Stewartia pseudocamellia extract may be fermented using lactic acid bacteria.

The lactic acid bacteria may be at least one selected from the group consisting of Lactobacillus plantarum (L. plantarum), Lacticaseibacillus rhamnosus (L. rhamnosus), and Lactiplantibacillus pentosus (L. pentosus) strains. The lactic acid bacteria may be used as strains for fermentation, but may also be included in an extract, which is a final product, and function.

In the present application, the term “fermented product” refers to the denaturation or modification of raw materials by physical or chemical methods using lactic acid bacteria, and refers to a decomposition process capable of obtaining the desired result in just a few hours or days, something that would take tens of thousands of years to transform in nature. Specifically, the fermented product may be a fermentation product produced by inoculating and fermenting a Stewartia pseudocamellia extract with lactic acid bacteria. The Stewartia pseudocamellia fermented product not only means a product obtained by fermenting a Stewartia pseudocamellia extract with lactic acid bacteria, but may also include a fermentation filtrate obtained by fermenting Stewartia pseudocamellia with lactic acid bacteria and then filtering the fermented Stewartia pseudocamellia, or a fermented extract obtained by extracting a Stewartia pseudocamellia fermented product with a solvent.

As used herein, the term “fermented extract” may refer to a useful substance itself produced by fermenting the Stewartia pseudocamellia extract using lactic acid bacteria, and may include all of a microbial culture medium in which lactic acid bacteria and a fermented extract coexist, a fermented extract obtained by centrifuging the culture medium to remove the lactic acid bacteria, a fermented extract obtained by filtering the lactic acid bacteria from the culture medium, a fermented extract obtained by sterilizing the lactic acid bacteria from the culture medium and filtering the sterilized lactic acid bacteria, a diluted solution obtained by diluting the fermented extract, a dried product obtained by drying the fermented extract, or the like. The process of fermenting and extracting Stewartia pseudocamellia may include extraction after fermentation or fermentation after extraction.

In one embodiment of the present application, in the inoculating of the extract with a lactic acid bacteria culture solution and the fermenting of the extract to obtain a fermented product, the fermentation temperature may be 25 to 40° C., 30 to 38° C., or 35 to 37° C. The fermentation time may be 1 to 6 days, 2 to 5 days, or 2 to 4 days.

The Stewartia pseudocamellia fermented product may be included in the composition at a concentration of 0.1 μg/ml to 200 μg/ml. More specifically, the concentration may be 0.5 μg/ml to 150 μg/ml, and even more specifically, 1 μg/ml to 100 μg/ml. When the Stewartia pseudocamellia fermented product is included at a concentration of less than 0.1 μg/ml, the effect is insignificant, and when the Stewartia pseudocamellia fermented product is included at a concentration of more than 200 μg/ml, it is possible to exhibit toxicity to other tissue cells in addition to cancer cells.

As used herein, the term “cancer” refers to or describes the physiological condition of mammals that are generally characterized by unregulated cell growth. “Cancer” refers to a condition in which a problem occurs in the regulatory functions of normal cell division, differentiation, and death, leading to abnormal and excessive proliferation, infiltration of surrounding tissues and organs, formation of masses, and destruction or deformation of existing structures.

The cancer may be at least one selected from the group consisting of breast cancer, pancreatic cancer, colorectal cancer, ovarian cancer, gastric cancer, cervical cancer, thyroid cancer, parathyroid cancer, lung cancer, non-small cell lung cancer, prostate cancer, gallbladder cancer, biliary tract cancer, bladder cancer, kidney cancer, melanoma, colon cancer, bone cancer, skin cancer, head and neck cancer, uterine cancer, rectal cancer, brain cancer, perianal cancer, fallopian tube carcinoma, endometrial carcinoma, vaginal cancer, vulvar carcinoma, esophageal cancer, small intestine cancer, endocrine gland cancer, adrenal cancer, soft tissue carcinoma, urethral cancer, penile cancer, ureteral cancer, renal cell carcinoma, renal pelvis carcinoma, blood cancer, acute myelogenous leukemia, myelodysplastic syndrome, chronic myelogenous leukemia, polycythemia vera, essential thrombocytosis, myelofibrosis, acute lymphoblastic leukemia, Hodgkin lymphoma, non-Hodgkin lymphoma, multiple myeloma, chronic lymphoblastic leukemia, central nervous system (CNS) tumors, spinal cord tumors, brainstem glioma, and pituitary adenoma.

The composition according to the present application may include a pharmaceutically effective amount of the Stewartia pseudocamellia fermented product alone, or may include one or more pharmaceutically acceptable carriers, excipients, or diluents. The pharmaceutically effective amount described above refers to an amount sufficient to prevent, ameliorate, and treat cancer.

In addition, the pharmaceutical composition described above refers to a composition that is physiologically acceptable and does not usually cause an allergic reaction such as a gastrointestinal disorder or vertigo or a reaction similar thereto when administered to a human. Examples of the carrier, the excipient, and the diluent include lactose, dextrose, sucrose, sorbitol, mannitol, xylitol, erythritol, maltitol, starch, acacia rubber, alginate, gelatin, calcium phosphate, calcium silicate, cellulose, methyl cellulose, polyvinyl pyrrolidone, water, methyl hydroxybenzoate, propyl hydroxybenzoate, talc, magnesium stearate, and mineral oil. Furthermore, the composition may additionally include a pharmaceutically acceptable filler, anti-agglomerating agent, lubricant, humectant, fragrance, emulsifier, preservative, analgesic, isotonic agent, antiseptic agent, other auxiliary agents, or and the like.

In the form of such pharmaceutical preparations, the present application may be administered in various oral and parenteral formulations during actual clinical administration. A solid preparation for oral administration may include a tablet, a pill, a powder, granules, a capsule, or the like. Further, a liquid preparation for oral administration includes a suspending agent, a liquid preparation for internal use, an emulsion, a syrup, or the like, and a preparation for parenteral administration (for example, intravenous, subcutaneous, intraperitoneal, or topical application) includes a sterilized aqueous solution, a non-aqueous solution, a suspension, an emulsion, a lyophilized preparation, or a suppository, but may be appropriately selected by those skilled in the art.

In addition, the composition of the present application may be formulated using a method known in the art so as to provide a rapid, sustained or delayed release of an active ingredient after administration to mammals. The formulation may be in the form of a powder, granules, a tablet, an emulsion, a syrup, an aerosol, a soft gelatin capsule, a hard gelatin capsule, a sterile injectable solution, a sterile powder, or the like.

The composition for preventing and treating cancer according to the present application may be administered via various routes including oral, transdermal, subcutaneous, intravenous or intramuscular, and the dosage of the active ingredient may be appropriately selected depending on various factors such as the administration route, the age, sex, body weight and severity of a patient. Furthermore, the composition for preventing and treating cancer of the present application may be administered in combination with a compound that has the effect of preventing, ameliorating, or treating cancer.

The present application provides a food composition for preventing or treating cancer, including a lactic acid bacteria fermented extract of Stewartia pseudocamellia as an active ingredient.

The food composition may include a health functional food. The “health functional food” is the same term as a food for special health use (FoSHU), and refers to a food with high medical and therapeutic effects, which is processed to efficiently exhibit bioregulatory functions in addition to providing nutrients. The “functional” means that useful effects for health applications such as regulating nutrients and physiological actions are obtained for the structure and function of the human body. The health functional food of the present application can be prepared by a method typically used in the art, and may be prepared by adding raw materials and components typically added in the art during the preparation of the health functional food. In addition, the preparation of the health functional food can also be prepared without limitation as long as it is recognized as a food. The health functional food composition of the present application can be prepared in various formulations and, unlike general medicines, is made from food ingredients, so there is an advantage in that the health functional food composition of the present application does not have the side effects that may occur when a drug is taken for a long period of time, and has excellent portability.

The form that the health functional food of the present application can take is not limited, may include all foods in a typical sense, and can be used interchangeably with terms known in the art, such as a functional food. Furthermore, the health functional food of the present application may be prepared by mixing other appropriate auxiliary ingredients and known additives that may be included in the food according to the selection of those skilled in the art. Examples of foods that may be added include various foods, beverages, gum, tea, vitamin complexes, functional foods, or the like. In addition, in the present application, the food includes special nutritional foods (for example, milk formulas, infant/baby foods, and the like), processed meat products, fish products, bean curd food, jellied foods, noodles (for example, ramens, noodles, and the like), breads, health supplements, seasonings (for example, soy sauce, soybean paste, red pepper paste, mixed soy paste, and the like), sauces, confectioneries (for example, snacks), candies, chocolates, gums, ice creams, processed dairy products (for example, fermented milk, cheese, and the like), other processed foods, kimchi, pickled foods (various types of kimchis, vegetables pickled in soy sauce, and the like), beverages (for example, fruit drinks, vegetable drinks, soybean milk, fermented beverages, and the like), natural seasonings (for example, a ramen soup base, and the like), or the like, but is not limited thereto. The food, beverage or food additive may be prepared by a conventional production method.

Furthermore, in addition to those described above, foods containing the composition for preventing and ameliorating cancer of the present application may contain various nutrients, vitamins, minerals (electrolytes), flavoring agents such as synthetic flavoring agents and natural flavoring agents, colorants, fillers (cheese, chocolate, and the like), pectic acid and salts thereof, alginic acid and salts thereof, organic acids, protective colloid thickeners, pH adjusting agents, stabilizers, antiseptic agents, glycerin, alcohols, carbonating agents used in a carbonated beverage, or the like, and the ingredients may be used independently or in combination.

Further, the health functional food composition of the present application may additionally include a physiologically acceptable carrier. The type of carrier is not particularly limited, and any carrier typically used in the art can be used. In addition, the health functional food composition may include, without limitation, additional ingredients that are typically used in food compositions and can improve the smell, taste, visual appearance, and the like, and may be selected and used in an appropriate amount depending on the type of food.

One embodiment of the present application provides a feed composition for preventing or ameliorating cancer, including a lactic acid bacteria fermented extract of Stewartia pseudocamellia or a fraction thereof as an active ingredient.

The feed composition may include a feed additive. The feed additive of the present application corresponds to supplementary feed under the Control of Livestock and Fish Feed Act.

As used herein, the “feed” may refer to any natural or artificial diet, meal, and the like, or a component of the meal for an animal to eat, ingest, and digest, or suitable for that.

The type of feed described above is not particularly limited, and any feed typically used in the art may be used. Non-limiting examples of the feed include vegetable feeds such as cereals, dried fruits, food processing by-products, algae, fibers, pharmaceutical by-products, fats and oils, starches, gourds or grain by-products; and animal feeds such as proteins, inorganic substances, fats and oils, minerals, fats and oils, single cell proteins, animal plankton or foods. These may be used either alone or in a mixture of two or more thereof.

As used herein, the term “feed additive” includes a substance added to feed for various purposes, such as supplementing nutrients and preventing body weight loss, increasing the digestibility of fiber in the feed, improving milk quality, preventing reproductive disorders and improving conception rates, and preventing heat stress in summer. The feed additive of the present application corresponds to supplementary feed under the Control of Livestock and Fish Feed Act of the Republic of Korea, and may additionally include a mineral preparation such as sodium bicarbonate, bentonite, magnesium oxide, and a complex mineral, a mineral preparation containing a trace mineral such as zinc, copper, cobalt, and selenium, a vitamin preparation such as carotene, vitamin A, vitamin D, vitamin E, nicotinic acid, and a vitamin B complex, a protected amino acid preparation such as methionine and lysine, a protected fatty acid preparation such as a fatty acid calcium salt, a live bacterial or yeast preparation such as a probiotic (lactic acid bacterial product), a yeast culture and a mold fermented product, and the like.

One embodiment of the present application provides a quasi-drug composition for preventing or ameliorating cancer, including a lactic acid bacteria fermented extract of Stewartia pseudocamellia or a fraction thereof as an active ingredient.

As used herein, the term “quasi-drug” refers to articles having a milder action than a pharmaceutical drug among articles used for the purpose of diagnosing, treating, ameliorating, alleviating, managing, or preventing human or animal diseases, and for example, in accordance with the Pharmaceutical Affairs Act of the Republic of Korea, the quasi-drug refers to an article used for pharmaceutical purposes.

The quasi-drug is an article that is a textile, a rubber product or a similar product used for the purpose of treating, relieving, managing, or preventing human or animal diseases, has a weak effect on the human body or does not act directly on the human body, is not an instrument or a machine, and is a similar product, and corresponds to one among the preparations used for sterilization, insecticidal and similar uses for prevention of infections, and refers to an article except for those which are not an instrument, a machine, or a device among articles used for the purpose of diagnosing, treating, relieving, managing, or preventing human or animal diseases, and is not an instrument, a machine, or a device among articles used for the purpose of pharmacologically affecting the structure and function of humans or animals, and also includes an external preparation and a personal hygiene product.

When the composition of the present application is used as a quasi-drug composition, the Stewartia pseudocamellia fermented product may be added as is or may be used appropriately together with other quasi-drug ingredients by a typical method. A mixing amount of the active ingredient may be determined appropriately according to the purpose of use.

The contents of the pharmaceutical composition or health functional food composition may be applied mutatis mutandis to the quasi-drug composition of the present application.

In addition, the composition may further include an additive such as a fragrance, a colorant, a bactericide, an antioxidant, an antiseptic agent, a moisturizer, a thickener, inorganic salts, an emulsifier, or a synthetic polymeric material to improve physical properties, but is not limited thereto.

One embodiment of the present application provides a method for preventing or treating cancer, the method including administering a pharmaceutical composition including a Stewartia pseudocamellia fermented product or a fraction thereof as an active ingredient to an individual other than a human.

As used herein, the term “individual” refers to all animals which are likely to develop or have developed cancer.

The feed composition according to the present application can be applied to any individual other than a human without limitation for the purpose of ameliorating, preventing or alleviating cancer. For example, the feed composition according to the present application can be applied to any individual such as a non-human animal such as a dog, a cat, a monkey, a rabbit, a guinea pig, a rat, a mouse, a cow, a sheep, a pig, or a goat, and a bird and a fish.

One embodiment of the present application provides a use of a composition including a lactic acid bacteria fermented extract of Stewartia pseudocamellia as an active ingredient for preparing a drug for preventing or treating cancer.

Since the lactic acid bacteria fermented extract of Stewartia pseudocamellia is the same as that described above, a detailed description thereof will be omitted.

Hereinafter, the present invention will be described in detail with reference to Examples and Comparative Examples. However, the following Examples and Comparative Examples are only for illustrating the present invention, and the content of the present invention is not limited by the following Examples.

### <Preparation Example> Preparation of Stewartia pseudocamellia Fermented Product

The leaves and stems of Stewartia pseudocamellia were dried in the shade and then ground into a powder. As the powder, a 100% powder from leaves and stems was used.

As the lactic acid bacteria, three lactic acid bacteria strains, i.e., Lactobacillus plantarum (KCTC 3105), Lacticaseibacillus rhamnosus (KACC 11953), and Lactiplantibacillus pentosus (KACC 17113), were acquired from the Korean Collection for Type Cultures (KCTC) and the Korean Agricultural Culture Collection (KACC) of the Rural Development Administration and used. The lactic acid bacteria strains were prepared in a medium according to the following Table 1 in an incubator adjusted to 37° C., autoclaved at 121° C. for 15 minutes, and then subcultured for 2 to 4 generations before use. After the strain was smeared on a sterilized solid medium, the strain was cultured for 24 hours, and inoculated into a liquid medium. The lactic acid bacteria culture solution, which had been cultured for 24 hours in an incubator adjusted to 37° C., was used for a Stewartia pseudocamellia fermented product.

TABLE 1
LactoBacilli MRS Broth (BD Difco ™ 288130)

| Component name | g/L |
|---|---|
| Proteose Peptone No. 3 | 10 |
| Beef extract | 10 |
| Yeast extract | 5 |
| Dextrose | 20 |
| Polysorbate 80 | 1 |
| Ammonium Citrate | 2 |
| Sodium Acetate | 5 |
| Magnesium Sulfate | 0.1 |
| Manganese Sulfate | 0.05 |
| Dipotassium Phosphate | 2 |

First, the Stewartia pseudocamellia powder and distilled water were mixed in a ratio of 1:20, and then an extract extracted with hot water at 100° C. for 6 hours was prepared. The hydrothermal extract was inoculated with each of the lactic acid bacteria culture solutions at 1% and then fermented at 37° C. for 48 hours. After fermentation, the extract was autoclaved at 121° C. for 15 minutes in order to inactivate the lactic acid bacteria strain, and then filtered through a 3 μm filter to prepare a fermented product. In this case, each of Examples 1 to 6 was prepared by varying the concentration of the fermented product.

### Comparative Example

The Stewartia pseudocamellia powder was mixed with 95% ethanol in a ratio of 1:20, and then the resulting mixture was extracted under reflux at 65° C. for 6 hours. After extraction, the extract was filtered through a 3 μm filter to prepare an extract, and the concentration of the extract was adjusted to 50 μg/ml to prepare Comparative Example 1.

Lactobacillus acidophilus (KACC 12419) was acquired from the Korean Agricultural Culture Collection (KACC) and used. The strain was prepared in a medium according to Table 1 above in an incubator adjusted to 37° C., autoclaved at 121° C. for 15 minutes, and then subcultured for 2 to 4 generations before use. After the strain was smeared on a sterilized solid medium, the strain was cultured for 24 hours, and inoculated into a liquid medium. The culture solution, which had been cultured for 24 hours in an incubator adjusted to 37° C., was used for a Stewartia pseudocamellia fermented product.

The method for preparing a Stewartia pseudocamellia fermented product was performed in the same manner as that described in the Preparation Example. In this case, L. acidophilus was used as a lactic acid bacterium, and Comparative Examples 2 and 3 were prepared by varying the concentration of the fermented product.

### <Experimental Example 1> Measurement of Killing Efficacy Against Blood Cancer Cells

### 1. Culture of Blood Cancer Cells (Molt-4)

Blood cancer cells (Molt-4, KCLB21582) were acquired from the Korea Cell Line Bank (KCLB, Seoul, Korea) and used. As a medium, RPMI 1640 (SH30027.01, Cytiva, USA) containing 10% Fetal Bovine Serum (FBS, Gibco, CA, USA), 25 mM HEPES (4-(2-hydroxyethyl)-1-piperazineethanesulfonic acid), and 25 mM NaHCO₃ was used. The cells were subcultured for 2 to 5 generations in an incubator adjusted to 95% humidity, 5% CO₂, and 37° C., and in this case, an antibiotic for a medium (Penicillin streptomycin, Gibco, CA, USA) was used to suppress microbial contamination or proliferation. For subculture of the cells, the cells were washed with phosphate-buffered saline (PBS, pH 7.4, Thermo Fisher Scientific, Massachusetts, USA) and then treated with trypsin-EDTA (Gibco, Thermo Fisher Scientific, Massachusetts, USA) to detach the cells, and then the cells were cultured by exchanging the medium every 48 to 72 hours.

### 2. Measurement of Molt-4 Cell Killing Efficacy

A Molt-4 cell line, Examples 1 to 3, and Comparative Examples 1 and 2 were aliquoted into 96-well plates and then cultured at 95% humidity, 5% CO₂, and 37° C. for 24 hours.

The viability of the cell line was measured using an MTS (CellTiter 96® Aqueous One Solution Cell Proliferation Assay, Promega, USA) reagent. After the cell line and the reagent were allowed to react at 37° C. for 60 minutes, absorbance was measured at 490 nm using a microplate reader (Molecular Devices, VersaMax ELISA Microplate Reader, USA). After the cells were treated with Examples 1 to 3 and Comparative Examples 1 and 2, cell viability was expressed as absorbance.

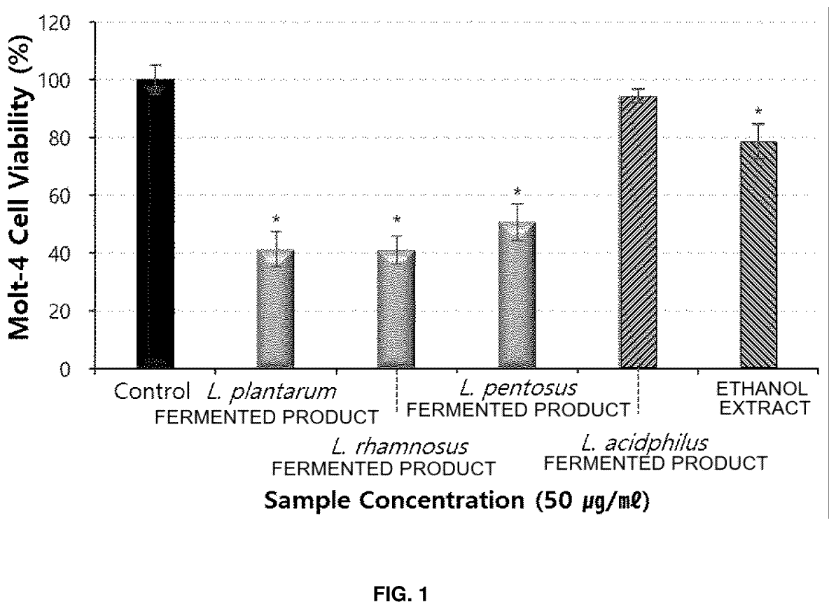

The results of the experiment on the killing efficacy of the Stewartia pseudocamellia fermented product and the ethanol extract on Molt-4 cells are shown in the following Table 2 and FIG. 1.

TABLE 2

| | | Concentration | Molt-4 cell |
|---|---|---|---|
| Classification | Strain | (μg/ml) | viability (%) |
| Control | — | — | 100.0 ± 2.5 |
| Example 1 | L. plantarum | 50 | 41.1 ± 6.1 |
| Example 2 | L. rhamnosus | 50 | 40.9 ± 4.9 |
| Example 3 | L. pentosus | 50 | 50.6 ± 6.4 |
| Comparative | | 50 | 78.4 ± 5.9 |
| Example 1 | | | |
| Comparative | L. acidophilus | 50 | 94.3 ± 2.4 |
| Example 2 | | | |

Referring to Table 2 and FIG. 1, all of Examples 1 to 3 showed the effect of killing blood cancer cells, and among them, Example 2, which is a Stewartia pseudocamellia fermented product using L. rhamnosus, had the best effect of preventing or treating blood cancer.

It can be confirmed that Comparative Examples 1 and 2 did not have good killing efficacy against Molt-4 cells.

### <Experimental Example 2> Measurement of Killing Efficacy Against Colorectal Cancer Cells

### 1. Culture of Colorectal Cancer Cells (HT-29)

Colorectal cancer cells (HT-29, KCLB 30038) were acquired from the Korea Cell Line Bank (KCLB, Seoul, Korea) and used. As a medium, RPMI 1640 (SH30027.01, Cytiva, USA) containing 10% Fetal Bovine Serum (FBS, Gibco, CA, USA), 25 mM HEPES, and 25 mM NaHCO₃ was used. The cells were subcultured for 2 to 5 generations in an incubator adjusted to 95% humidity, 5% CO₂, and 37° C., and in this case, an antibiotic for a medium (Penicillin streptomycin, Gibco, CA, USA) was used to suppress microbial contamination or proliferation. For subculture of the cells, the cells were washed with phosphate-buffered saline (PBS, pH 7.4, Thermo Fisher Scientific, Massachusetts, USA) and then treated with trypsin-EDTA (Gibco, Thermo Fisher Scientific, Massachusetts, USA) to detach the cells, and then the cells were cultured by exchanging the medium every 48 to 72 hours.

### 2. Measurement of HT-29 Cell Killing Efficacy

An HT-29 cell line, Examples 1 to 3, and Comparative Examples 1 and 2 were aliquoted into 96-well plates and then cultured at 95% humidity, 5% CO₂, and 37° C. for 24 hours.

The viability of the cell line was measured using an MTS (CellTiter 96® Aqueous One Solution Cell Proliferation Assay, Promega, USA) reagent. After the cell line and the reagent were allowed to react at 37° C. for 60 minutes, absorbance was measured at 490 nm using a microplate reader (Molecular Devices, VersaMax ELISA Microplate Reader, USA). After the cells were treated with Examples 1 to 3 and Comparative Example 2, cell viability was expressed as absorbance.

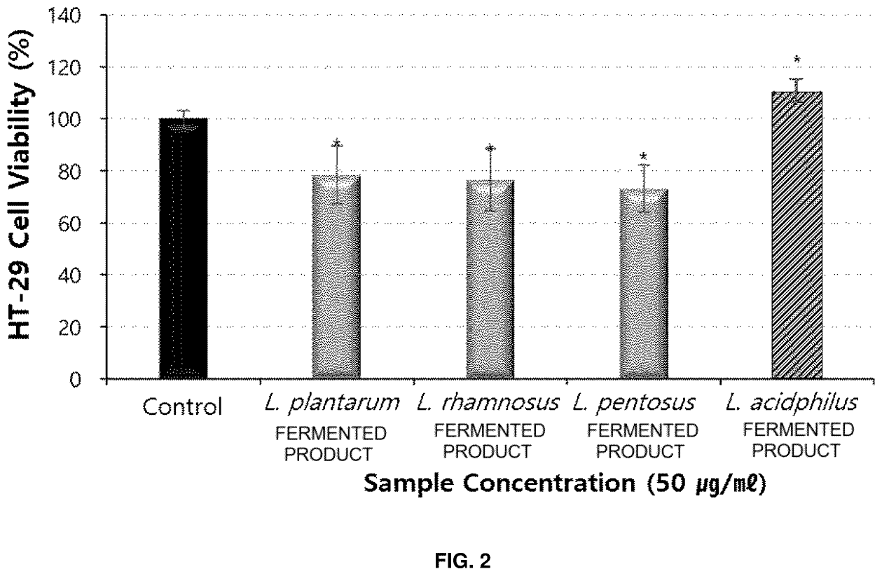

The results of the experiment on the killing efficacy of the Stewartia pseudocamellia fermented product and the ethanol extract on HT-29 cells are shown in the following Table 3 and FIG. 2.

TABLE 3

| | | Concentration | HT-29 cell |
|---|---|---|---|
| Classification | Strain | (μg/ml) | viability (%) |
| Control | — | — | 100.0 ± 3.1 |
| Example 1 | L. plantarum | 50 | 78.4 ± 11.1 |
| Example 2 | L. rhamnosus | 50 | 76.5 ± 11.9 |
| Example 3 | L. pentosus | 50 | 73.3 ± 9.1 |
| Comparative | L. acidophilus | 50 | 110.7 ± 4.5 |
| Example 2 | | | |

Referring to Table 3 and FIG. 2, all of Examples 1 to 3 showed the effect of killing colorectal cancer cells, and among them, Example 3, which is a Stewartia pseudocamellia fermented product using L. pentosus, had the best effect of preventing or treating colorectal cancer.

In contrast, in the case of Comparative Example 2, colorectal cancer cells proliferated instead.

### <Experimental Example 3> Measurement of Killing Efficacy Against Breast Cells

### 1. Culture of Breast Cancer Cells (MCF-7)

Breast cancer cells (MCF-7, KCLB30022) were acquired from the Korea Cell Line Bank (KCLB, Seoul, Korea) and used. As a medium, a minimum essential medium (SH30024.01, Cytiva, USA) containing 10% Fetal Bovine Serum (FBS, Gibco, CA, USA), 25 mM HEPES, and 25 mM NaHCO₃ was used. The cells were subcultured for 2 to 5 generations in an incubator adjusted to 95% humidity, 5% CO₂, and 37° C., and in this case, an antibiotic for a medium (Penicillin streptomycin, Gibco, CA, USA) was used to suppress microbial contamination or proliferation. For subculture of the cells, the cells were washed with phosphate-buffered saline (PBS, pH 7.4, Thermo Fisher Scientific, Massachusetts, USA) and then treated with trypsin-EDTA (Gibco, Thermo Fisher Scientific, Massachusetts, USA) to detach the cells, and then the cells were cultured by exchanging the medium every 48 to 72 hours.

### 2. Measurement of MCF-7 Cell Killing Efficacy

An MCF-7 cell line, Examples 4 to 6, and Comparative Example 4 were aliquoted into 96-well plates and then cultured at 95% humidity, 5% CO₂, and 37° C. for 24 hours.

The viability of the cell line was measured using an MTS (CellTiter 96 ® Aqueous One Solution Cell Proliferation Assay, Promega, USA) reagent. After the cell line and the reagent were allowed to react at 37° C. for 60 minutes, absorbance was measured at 490 nm using a microplate reader (Molecular Devices, VersaMax ELISA Microplate Reader, USA). After the cells were treated with Examples 4 to 6 and Comparative Example 3, cell viability was expressed as absorbance.

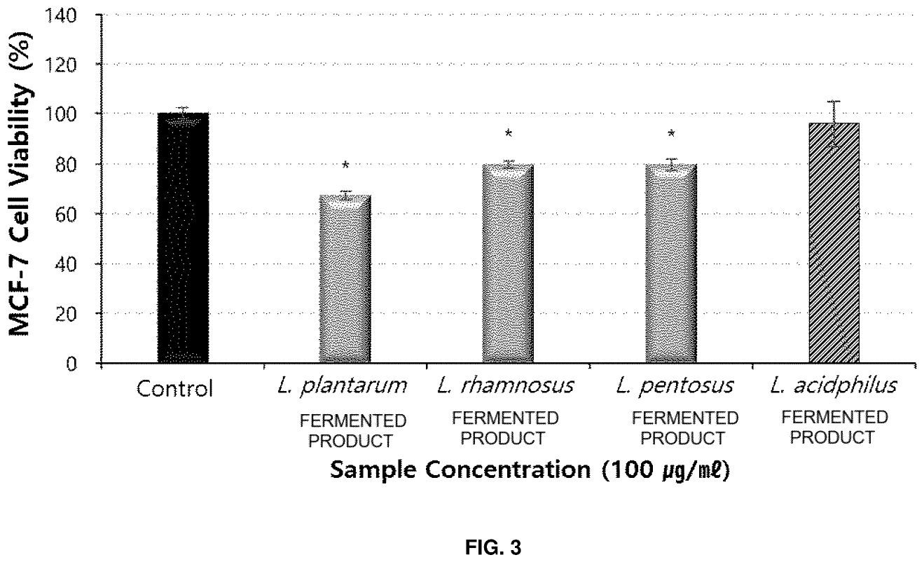

The results of the experiment on the killing efficacy of the Stewartia pseudocamellia fermented product and the ethanol extract on MCF-7 cells are shown in the following Table 4 and FIG. 3.

TABLE 4

| | | Concentration | MCF-7 cell |
|---|---|---|---|
| Classification | Strain | (μg/ml) | viability (%) |
| Control | — | — | 100.0 ± 2.3 |
| Example 4 | L. plantarum | 100 | 67.2 ± 1.6 |
| Example 5 | L. rhamnosus | 100 | 79.6 ± 1.4 |
| Example 6 | L. pentosus | 100 | 79.5 ± 2.3 |
| Comparative | L. acidophilus | 100 | 96.1 ± 9.1 |
| Example 3 | | | |

Referring to Table 4 and FIG. 3, all of Examples 4 to 6 showed the effect of killing breast cancer cells, and among them, Example 4, which is a Stewartia pseudocamellia fermented product using L. plantarum, had the best effect of preventing or treating breast cancer.

It can be confirmed that in the case of Comparative Example 3, the killing efficacy against MCF-7 cells is lower than that of the Example.

### <Experimental Example 4> Measurement of Killing Efficacy Against Pancreatic Cancer Cells

### 1. Culture of Pancreatic Cancer Cells (MIA PaCa-2)

Pancreatic cancer cells (MIA PaCa-2, KCLB 21420) were acquired from the Korea Cell Line Bank (KCLB, Seoul, Korea) and used. As a medium, DMEM High Glucose (SH30243.01, Cytiva, USA) containing a 10% Fetal Bovine Serum (FBS, Gibco, CA, USA) was used. The cells were subcultured for 2 to 5 generations in an incubator adjusted to 95% humidity, 5% CO₂, and 37° C., and in this case, an antibiotic for a medium (Penicillin streptomycin, Gibco, CA, USA) was used to suppress microbial contamination or proliferation. For subculture of the cells, the cells were washed with phosphate-buffered saline (PBS, pH 7.4, Thermo Fisher Scientific, Massachusetts, USA) and then treated with trypsin-EDTA (Gibco, Thermo Fisher Scientific, Massachusetts, USA) to detach the cells, and then the cells were cultured by exchanging the medium every 48 to 72 hours.

### 2. Measurement of MIA PaCa-2 Cell Killing Efficacy

An MIA PaCa-2 cell line, Examples 4 to 6, and Comparative Example 3 were aliquoted into 96-well plates and then cultured at 95% humidity, 5% CO₂, and 37° C. for 24 hours.

The viability of the cell line was measured using an MTS (CellTiter 96® Aqueous One Solution Cell Proliferation Assay, Promega, USA) reagent. After the cell line and the reagent were allowed to react at 37° C. for 60 minutes, absorbance was measured at 490 nm using a microplate reader (Molecular Devices, VersaMax ELISA Microplate Reader, USA). After the cells were treated with Examples 4 to 6 and Comparative Example 3, cell viability was expressed as absorbance.

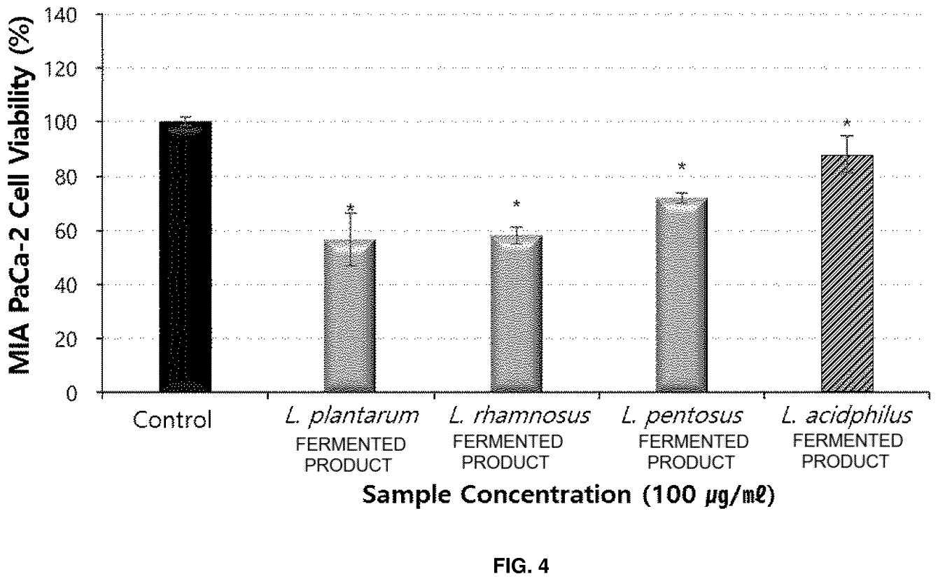

The results of the experiment on the killing efficacy of the Stewartia pseudocamellia fermented product and the ethanol extract on MIA PaCa-2 cells are shown in the following Table 5 and FIG. 4.

TABLE 5

| | | Concentration | MIA PaCa-2 cell |
|---|---|---|---|
| Classification | Strain | (μg/ml) | viability (%) |
| Control | — | — | 100.0 ± 1.7 |
| Example 4 | L. plantarum | 100 | 56.6 ± 9.6 |
| Example 5 | L. rhamnosus | 100 | 58.1 ± 3.1 |
| Example 6 | L. pentosus | 100 | 72.0 ± 1.9 |
| Comparative | L. acidophilus | 100 | 87.8 ± 6.7 |
| Example 3 | | | |

Referring to Table 5 and FIG. 4, all of Examples 4 to 6 showed the effect of killing pancreatic cancer cells, and among them, Example 4, which is a Stewartia pseudocamellia fermented product using L. plantarum, had the best effect of preventing or treating pancreatic cancer.

It can be confirmed that Comparative Example 3 did not have good killing efficacy against MIA PaCa-2 cells.

Although specific parts of the present application have been described in detail, it is obvious to those skilled in the art that such specific descriptions are only preferred embodiments and the scope of the present application is not limited thereby. Accordingly, the substantial scope of the present application will be defined by the appended claims and equivalents thereof. Furthermore, various modifications and improvements made by those skilled in the art using the basic concept of the present application as defined in the claims also fall within the scope of the present application.

1. A method for preparing a pharmaceutical composition for preventing or treating cancer, the method comprising:
preparing a Stewartia pseudocamellia extract; and
inoculating the extract with a lactic acid bacteria culture solution and fermenting the extract to obtain a fermented product,
wherein the pharmaceutical composition comprises the fermented product as an active ingredient.

2. The method of claim 1, wherein the lactic acid bacteria is at least one selected from the group consisting of Lactobacillus plantarum, Lacticaseibacillus rhamnosus, and Lactiplantibacillus pentosus.

3. The method of claim 1, wherein in the obtaining of the fermented product, the fermentation is performed at 30° C. to 37° C. for 1 day to 6 days.

4. The method of claim 1, wherein the preparing of the Stewartia pseudocamellia extract extracts the extract by mixing a Stewartia pseudocamellia powder and a solvent in a ratio of 1:5 to 1:30.

5. The method of claim 1, wherein the Stewartia pseudocamellia is one or more of leaves and stems, and the extract is an extract extracted with hot water at 80° C. to 110° C. for 2 to 12 hours.

6. The method of claim 1, wherein the cancer is at least one selected from the group consisting of breast cancer, pancreatic cancer, colorectal cancer, ovarian cancer, gastric cancer, cervical cancer, thyroid cancer, parathyroid cancer, lung cancer, non-small cell lung cancer, prostate cancer, gallbladder cancer, biliary tract cancer, bladder cancer, kidney cancer, melanoma, colon cancer, bone cancer, skin cancer, head and neck cancer, uterine cancer, rectal cancer, brain cancer, perianal cancer, fallopian tube carcinoma, endometrial carcinoma, vaginal cancer, vulvar carcinoma, esophageal cancer, small intestine cancer, endocrine gland cancer, adrenal cancer, soft tissue carcinoma, urethral cancer, penile cancer, ureteral cancer, renal cell carcinoma, renal pelvis carcinoma, blood cancer, acute myelogenous leukemia, myelodysplastic syndrome, chronic myelogenous leukemia, polycythemia vera, essential thrombocytosis, myelofibrosis, acute lymphoblastic leukemia, Hodgkin lymphoma, non-Hodgkin lymphoma, multiple myeloma, chronic lymphoblastic leukemia, central nervous system (CNS) tumors, spinal cord tumors, brainstem glioma, and pituitary adenoma.

7. A pharmaceutical composition prepared by the method of claim 1 and comprising a lactic acid bacteria fermented extract of Stewartia pseudocamellia as an active ingredient.

8.-10. (canceled)

11. A method for treating cancer, the method comprising:
administering to a sublet in need thereof the pharmaceutical composition of claim 1.

12. (canceled)

13. The method of claim 11, wherein the cancer is at least one selected from the group consisting of breast cancer, pancreatic cancer, colorectal cancer, ovarian cancer, gastric cancer, cervical cancer, thyroid cancer, parathyroid cancer, lung cancer, non-small cell lung cancer, prostate cancer, gallbladder cancer, biliary tract cancer, bladder cancer, kidney cancer, melanoma, colon cancer, bone cancer, skin cancer, head and neck cancer, uterine cancer, rectal cancer, brain cancer, perianal cancer, fallopian tube carcinoma, endometrial carcinoma, vaginal cancer, vulvar carcinoma, esophageal cancer, small intestine cancer, endocrine gland cancer, adrenal cancer, soft tissue carcinoma, urethral cancer, penile cancer, ureteral cancer, renal cell carcinoma, renal pelvis carcinoma, blood cancer, acute myelogenous leukemia, myelodysplastic syndrome, chronic myelogenous leukemia, polycythemia vera, essential thrombocytosis, myelofibrosis, acute lymphoblastic leukemia, Hodgkin lymphoma, non-Hodgkin lymphoma, multiple myeloma, chronic lymphoblastic leukemia, central nervous system (CNS) tumors, spinal cord tumors, brainstem glioma, and pituitary adenoma.
